(19) United States
(12) Patent Application Publication
(10) Pub. No.: US 2017/0367972 A1
(43) Pub. Date: Dec. 28, 2017

(54) Formulations and Methods of Treatment for Treating Nail Fungal Infections

(71) Applicant: Stephen Brodsky, Malvern, PA (US)

(72) Inventor: Stephen Brodsky, Malvern, PA (US)

(21) Appl. No.: 15/632,463

(22) Filed: Jun. 26, 2017

Related U.S. Application Data
(60) Provisional application No. 62/493,273, filed on Jun. 27, 2016.

(51) Int. Cl. A61K 9/00; C12Q 1/04; A61K 9/06; C12Q 1/68

(52) U.S. Cl. CPC A61K 9/0014; C12Q 1/6895; C12Q 1/04; A61K 9/06; C12Q 1/6809; C12Q 2600/106; C12Q 2600/112

(57) ABSTRACT

The present invention relates to methods and compositions for the treatment of infections of the nails, in particular the treatment of onychomycosis.

### RELATED APPLICATIONS

This application claims priority to U.S. Ser. No. 62/493,273 filed on Jun. 27, 2016, which is hereby incorporated into this application in its entirety.

### FIELD OF THE INVENTION

The present invention relates to methods and compositions for the treatment of infections of the nails, in particular the treatment of onychomycosis.

### BACKGROUND OF THE INVENTION

Fungal infection of the toenails or fingernails is caused by a fungal microbe that invades the nail bed. Fungal nail infection is also termed onychomycosis and tinea unguium. Fungal nail infection causes fingernails or toenails to thicken, discolor, disfigure, and split. At first, onychomycosis appears to be only a cosmetic concern. Without treatment, however, the toenails can become so thick that they press against the inside of the shoes, causing pressure, irritation, and pain. Fingernail infection may cause psychological, social, or employment-related problems.

Half of all nail disorders are caused by onychomycosis, and it is the most common nail disease in adults. Toenails are much more likely to be infected than fingernails. The incidence of onychomycosis has been increasing and is associated with diabetes suppressed immune system, and increasing age. Adults, especially the elderly, are more likely to have onychomycosis than children. Most cutaneous infections are the work of the homogeneous group of keratinophilic fungi known as dermatophytes. For quite some time the dermatophyte Trichophyton rubrum has been considered the major cause of tinea pedis and onychomycosis. With the recent onset of DNA sequencing technology, it is becoming evident that rarely is a single fungus present in and around the nail and in addition to multiple fungal species, multiple bacteria also exist in the nail and raise the suspicion that a multispecies biofilm in and under the nail plate is the source of the resistance of onychomycosis to oral and topical therapies. Dermatophytoses of the fingernails and toenails, in contrast to those at other body sites, are particularly difficult to eradicate with drug treatment that has traditionally been focused on a small number of common pathogens. Some of the resistance is the consequence of factors intrinsic to the nail—the hard, protective nail plate, sequestration of pathogens between the nail bed and plate, and slow growth of the nail. In addition the presence of a multispecies biofilm, as well as the relatively poor efficacy of available pharmacologic agents may be an additional cause.

Although there are a variety of treatments available, there appears to be no current cure for onychomycosis and none of these are directed specifically at the bacteria present. Treatments such as Sporonox, Lamisil and Diflucan contain active ingredients such as itraconazole, terbinafine, ciclopirox an fluconazole but the medications do not eradicate the problem for many patients. Such treatments often take months and depend on the nail bed growing out completely. Therefore, there is a need for mediations that will effectively treat what we will term the onychomycobacterial OMB infection in a timely fashion.

### SUMMARY OF THE INVENTION

The present invention relates to compositions for the treatment of OMB where the composition has specific antibiotic(s) and antifungal agent(s) in a tissue penetrating agent. The present invention relates to compositions having at least one antibiotic and at least one antifungal agent in a tissue penetrating agent, hydrogen peroxide or an additional antiseptic agent, and a non-steroidal anti-inflammatory agent to reduce the tissue inflammation essential to the life of a biofilm. The compositions of the present invention include compositions where the antifungal agents include but are not limited to, itraconazole and fluconazole and the antibiotics are vancomycin, levofloxacin and mupirocin and the NSAID ibuprofen. The present invention also includes compositions containing about 0.5 wt % to about 1.5 wt % itraconazole, about 0.5 wt % to about 1.5 wt % fluconazole, about 4 wt % to about 5 wt %, vancomycin, about 1 wt % to about 3 wt %, levofloxacin, about 3 wt % to about 5 wt % mupirocin is and about 1 wt % to about 3 wt % ibuprofen and the tissue penetrating agent, DMSO. Compositions of the present invention also include 1 wt % itraconazole, 1 wt % fluconazole, 5 wt % vancomycin, 2 wt % levofloxacin, 4 wt % mupirocin and 2 wt % ibuprofen in DMSO. The present invention also relates to compositions for the treatment of onychomycosis comprising an antiseptic such as hydrogen peroxide and other alternative antibiotics or antimicrobial agents that may be identified as necessary by the results of accumulating DNA data. In some compositions the may be the antibiotics vancomycin, clindamycin, or additional antibiotics. In compositions containing vancomycin or clindamycin they may be present in an amounts from 1-5 wt %. If hydrogen peroxide is present it may be present in concentrations of 20 to about 30%.

The present invention also relates to methods of treating OMB by administering the compositions of the present invention to the nail(s) of afflicted patients. The administration of the compositions may be conducted daily or multiple times daily and the duration of treatment may last a week to several months.

The present invention also relates to methods of determining the composition of the various etiological agents that cause OMB in a given patient by determining the presence of particular fungal or bacterial species by various biochemical techniques including restriction fragment length polymorphism (RFLP), Real time PCR and multiplex PCR assay.

### DETAILED DESCRIPTION OF THE INVENTION

The compositions of the present invention contain at least one antifungal medicament. Antifungal active compounds include but are not limited to polyene antifungal drugs, azole antifungal drugs, allylamine and morpholine antifungal drugs and antimetabolite antifungal drugs.

The polyene compounds are so named because of the alternating conjugated double bonds that constitute a part of their macrolide ring structure. The polyene antibiotics are all products of Streptomyces species. These drugs interact with sterols in cell membranes (ergosterol in fungal cells; cholesterol in human cells) to form channels through the membrane, causing the cells to become leaky. The polyene antifungal agents include nystatin, amphotericin B, and pimaricin. Amphotericin, nystatin, and pimaricin interact with sterols in the cell membrane (ergosterol in fungi, cholesterol in humans) to form channels through which small molecules leak from the inside of the fungal cell to the outside.

The azole antifungal agents have five-membered organic rings that contain either two or three nitrogen molecules (the imidazoles and the triazoles respectively). The clinically useful imidazoles are clotrimazole, miconazole, and ketoconazole. Two important triazoles are itraconazole and fluconazole. In general, the azole antifungal agents are thought to inhibit cytochrome P₄₅₀-dependent enzymes involved in the biosynthesis of cell membrane sterols. Fluconazole, itraconazole, and ketoconazole inhibit cytochrome P₄₅₀-dependent enzymes (particularly C14-demethylase) involved in the biosynthesis of ergosterol, which is required for fungal cell membrane structure and function.

Allylamines (naftifine, terbinafine) inhibit ergosterol biosynthesis at the level of squalene epoxidase. The morpholine drug, amorolfine, inhibits the same pathway at a later step.

In contrast to the situation with antibacterial agents, few antimetabolites are available for use against fungi. The best example is 5-fluorocytosine, a fluorinated analog of cytosine. It inhibits both DNA and RNA synthesis via intracytoplasmic conversion to 5-fluorouracil, 5-Fluorocytosine acts as an inhibitor of both DNA and RNA synthesis via the intracytoplasmic conversion of 5-fluorocytosine to 5-fluorouracil.

Griseofulvin is an antifungal antibiotic produced by Penicillium griseofulvum. It is active in vitro against most dermatophytes and has been the drug of choice for chronic infections caused by these fungi (e.g., nail infections with Trichophyton rubrum) since it is orally administered and presumably incorporated into actively growing tissue. It is still used in such instances but is being challenged by some of the newer azole antifungal agents. The drug inhibits mitosis in fungi. Another antifiungal agent is undecylenic acid

The amount of each antifungal compound in the compositions of the present invention may be about 0.5 wt %, 0.6 wt % 0.7 wt %, 0.8 wt %, 0.9 wt %, 1.0 wt %, 1.1 wt % 1.2 wt %, 1.3 wt %, 1.4 wt %, 1.5 wt %, 1.6 wt %, 1.7 wt %, 1.8 wt %, 1.9 wt %, 2.0 wt %, 2.1 wt %, 2.2 wt %, 2.3 wt %, 2.4 wt %, 2.5 wt %, 2.6 wt %, 2.7 wt %, 2.8 wt %, 2.9 wt %, 3.0 wt %, 3.1 wt %, 3.2 wt %, 3.3 wt %, 3.4 wt %, 3.5 wt %, 3.6 wt %, 3.7 wt %, 3.8 wt %, 3.9 wt %, 4.0 wt %, 4.1 wt %, 4.2 wt %, 4.3 wt %, 4.4 wt %, 4.5 wt %, 4.6 wt %, 4.7 wt %, 4.8 wt %, 4.9 wt %, 5.0 wt %, 5.1 wt %, 5.2 wt %, 5.3 wt %, 5.4 wt %, 5.5 wt %, 5.6 wt %, 5.7 wt %, 5.8 wt %, 5.9 wt %, 6.0 wt %, 6.1 wt %, 6.2 wt %, 6.3 wt %, 6.4 wt %, 6.5 wt %, 6.6 wt %, 6.7 wt %, 6.8 wt %, 6.9 wt %, 7.0 wt %.

The amount of each antifungal compound in the compositions of the present invention may be between about 0.1 wt % to about 5.0 wt %, or about 0.1 wt % to about 4.5 wt %, or about 0.1 wt % to about 4.0 wt %, or about 0.1 wt % to about 3.5 wt %, or about 0.1 wt % to about 3.0 wt %, or about 0.1 wt % to about 2.5 wt %, or about 0.1 wt % to about 2.0 wt %, or about 0.1 wt % to about 1.5 wt %, or about 0.1 wt % to about 1.0 wt %, or about 0.1 wt % to about 0.5 wt %, or about 0.1 wt % to about 0.3 wt %, or about 0.3 wt % to about 5.0 wt %, or about 0.3 wt % to about 4.5 wt %, or about 0.3 wt % to about 4.0 wt %, or about 0.3 wt % to about 3.5 wt %, or about 0.3 wt % to about 3.0 wt %, or about 0.3 wt % to about 2.5 wt %, or about 0.3 wt % to about 2.0 wt %, or about 0.3 wt % to about 1.5 wt %, or about 0.3 wt % to about 1.0 wt %, or about 0.3 wt % to about 0.5 wt %, or about 0.5 wt % to about 5.0 wt %, or about 0.5 wt % to about 4.5 wt %, or about 0.5 wt % to about 4.0 wt %, or about 0.5 wt % to about 3.5 wt %, or about 0.5 wt % to about 3.0 wt %, or about 0.5 wt % to about 2.5 wt %, or about 0.5 wt % to about 2.0 wt %, or about 0.5 wt % to about 1.5 wt %, or about 0.5 wt % to about 1.0 wt %, or about 0.7 wt % to about 5.0 wt %, or about 0.7 wt % to about 4.5 wt %, or about 0.7 wt % to about 4.0 wt %, or about 0.7 wt % to about 3.5 wt %, or about 0.7 wt % to about 3.0 wt %, or about 0.7 wt % to about 2.5 wt %, or about 0.7 wt % to about 2.0 wt %, or about 0.7 wt % to about 1.5 wt %, or about 0.7 wt % to about 1.0 wt %, or about 1.0 wt % to about 5.0 wt %, or about 1.0 wt % to about 4.5 wt %, or about 1.0 wt % to about 4.0 wt %, or about 1.0 wt % to about 3.5 wt %, or about 1.0 wt % to about 3.0 wt %, or about 1.0 wt % to about 2.5 wt %, or about 1.0 wt % to about 2.0 wt %, or about 1.0 wt % to about 1.5 wt %, or about 1.25 wt % to about 5.0 wt %, or about 1.25 wt % to about 4.5 wt %, or about 1.25 wt % to about 4.0 wt %, or about 1.25 wt % to about 3.5 wt %, or about 1.25 wt % to about 3.0 wt %, or about 1.25 wt % to about 2.5 wt %, or about 1.25 wt % to about 2.0 wt %, or about 1.25 wt % to about 1.5 wt %, or about 1.5 wt % to about 5.0 wt %, or about 1.5 wt % to about 4.5 wt %, or about 1.5 wt % to about 4.0 wt %, or about 1.5 wt % to about 3.5 wt %, or about 1.5 wt % to about 3.0 wt %, or about 1.5 wt % to about 2.5 wt %, or about 1.5 wt % to about 2.0 wt %, or about 1.75 wt % to about 5.0 wt %, or about 1.75 wt % to about 4.5 wt %, or about 1.75 wt % to about 4.0 wt %, or about 1.75 wt % to about 3.5 wt %, or about 1.75 wt % to about 3.0 wt %, or about 1.75 wt % to about 2.5 wt %, or about 1.75 wt % to about 2.0 wt %, or about 2.0 wt % to about 5.0 wt %, or about 2.0 wt % to about 4.5 wt %, or about 2.0 wt % to about 4.0 wt %, or about 2.0 wt % to about 3.5 wt %, or about 2.0 wt % to about 3.0 wt %, or about 2.0 wt % to about 2.5 wt %, or about 2.5 wt % to about 4.5 wt %, or about 2.5 wt % to about 4.0 wt %, or about 2.5 wt % to about 3.5 wt %, or about 2.5 wt % to about 3.0 wt %.

The compositions of the present invention contain at least one antibacterial agent. Antibacterial active compounds include but are not limited to Aminoglycosides, Cephalosporins, Fluoroquinolones, Macrolides, Penicillins, Sulfonamides, Tetracyclines Also included are glycopeptide antibiotics such as Vancomycin, Teicoplanin, Bleomycin, Vancomycin Hydrochloride as well as the fluoroquinolone antibiotics such as Levofloxacin, ciprofloxacin, moxifloxacin, and ofloxacin. Other antibiotics include mupirocin and clindamycin and chemical antibiotics including chloroxylenol.

The amount of each antibacterial compound in the compositions of the present invention may be about 0.5 wt %, 0.6 wt % 0.7 wt %, 0.8 wt %, 0.9 wt %, 1.0 wt %, 1.1 wt % 1.2 wt %, 1.3 wt %, 1.4 wt %, 1.5 wt %, 1.6 wt %, 1.7 wt %, 1.8 wt %, 1.9 wt %, 2.0 wt %, 2.1 wt %, 2.2 wt %, 2.3 wt %, 2.4 wt %, 2.5 wt %, 2.6 wt %, 2.7 wt %, 2.8 wt %, 2.9 wt %, 3.0 wt %, 3.1 wt %, 3.2 wt %, 3.3 wt %, 3.4 wt %, 3.5 wt %, 3.6 wt %, 3.7 wt %, 3.8 wt %, 3.9 wt %, 4.0 wt %, 4.1 wt %, 4.2 wt %, 4.3 wt %, 4.4 wt %, 4.5 wt %, 4.6 wt %, 4.7 wt %, 4.8 wt %, 4.9 wt %, 5.0 wt %, 5.1 wt %, 5.2 wt %, 5.3 wt %, 5.4 wt %, 5.5 wt %, 5.6 wt %, 5.7 wt %, 5.8 wt %, 5.9 wt %, 6.0 wt %, 6.1 wt %, 6.2 wt %, 6.3 wt %, 6.4 wt %, 6.5 wt %, 6.6 wt %, 6.7 wt %, 6.8 wt %, 6.9 wt %, 7.0 wt %.

The amount of each antibacterial compound in the compositions of the present invention may be between about 0.1 wt % to about 7.0 wt %, or about 0.1 wt % to about 6.5 wt %, or about 0.1 wt % to about 6.0 wt %, or about 0.1 wt % to about 5.5 wt %, or about 0.1 wt % to about 5.0 wt %, or about 0.1 wt % to about 4.5 wt %, or about 0.1 wt % to about 4.0 wt %, or about 0.1 wt % to about 3.5 wt %, or about 0.1 wt % to about 3.0 wt %, or about 0.1 wt % to about 2.5 wt %, or about 0.1 wt % to about 2.0 wt %, or about 0.1 wt % to about 1.5 wt %, or about 0.1 wt % to about 1.0 wt %, or about 0.1 wt % to about 0.5 wt %, or about 0.1 wt % to about 0.3 wt %, or about 0.3 wt % to about 7.0 wt %, or about 0.3 wt % to about 6.5 wt %, or about 0.3 wt % to about 6.0 wt %, or about 0.3 wt % to about 5.5 wt %, or about 0.3 wt % to about 5.0 wt %, or about 0.3 wt % to about 4.5 wt %, or about 0.3 wt % to about 4.0 wt %, or about 0.3 wt % to about 3.5 wt %, or about 0.3 wt % to about 3.0 wt %, or about 0.3 wt % to about 2.5 wt %, or about 0.3 wt % to about 2.0 wt %, or about 0.3 wt % to about 1.5 wt %, or about 0.3 wt % to about 1.0 wt %, or about 0.3 wt % to about 0.5 wt %, or about 0.5 wt % to about 7.0 wt %, or about 0.5 wt % to about 6.5 wt %, or about 0.5 wt % to about 6.0 wt %, or about 0.5 wt % to about 5.5 wt %, or about 0.5 wt % to about 5.0 wt %, or about 0.5 wt % to about 4.5 wt %, or about 0.5 wt % to about 4.0 wt %, or about 0.5 wt % to about 3.5 wt %, or about 0.5 wt % to about 3.0 wt %, or about 0.5 wt % to about 2.5 wt %, or about 0.5 wt % to about 2.0 wt %, or about 0.5 wt % to about 1.5 wt %, or about 0.5 wt % to about 1.0 wt %, or about 0.7 wt % to about 7.0 wt %, or about 0.7 wt % to about 6.5 wt %, or about 0.7 wt % to about 6.0 wt %, or about 0.7 wt % to about 5.5 wt %, or about 0.7 wt % to about 5.0 wt %, or about 0.7 wt % to about 4.5 wt %, or about 0.7 wt % to about 4.0 wt %, or about 0.7 wt % to about 3.5 wt %, or about 0.7 wt % to about 3.0 wt %, or about 0.7 wt % to about 2.5 wt %, or about 0.7 wt % to about 2.0 wt %, or about 0.7 wt % to about 1.5 wt %, or about 0.7 wt % to about 1.0 wt %, or about 1.0 wt % to about 7.0 wt %, or about 1.0 wt % to about 6.5 wt %, or about 1.0 wt % to about 6.0 wt %, or about 1.0 wt % to about 5.5 wt %, or about 1.0 wt % to about 5.0 wt %, or about 1.0 wt % to about 4.5 wt %, or about 1.0 wt % to about 4.0 wt %, or about 1.0 wt % to about 3.5 wt %, or about 1.0 wt % to about 3.0 wt %, or about 1.0 wt % to about 2.5 wt %, or about 1.0 wt % to about 2.0 wt %, or about 1.0 wt % to about 1.5 wt %, or about 1.25 wt % to about 7.0 wt %, or about 1.25 wt % to about 6.5 wt %, or about 1.25 wt % to about 6.0 wt %, or about 1.25 wt % to about 5.5 wt %, or about 1.25 wt % to about 5.0 wt %, or about 1.25 wt % to about 4.5 wt %, or about 1.25 wt % to about 4.0 wt %, or about 1.25 wt % to about 3.5 wt %, or about 1.25 wt % to about 3.0 wt %, or about 1.25 wt % to about 2.5 wt %, or about 1.25 wt % to about 2.0 wt %, or about 1.25 wt % to about 1.5 wt %, or about 1.5 wt % to about 7.0 wt %, or about 1.5 wt % to about 6.5 wt %, or about 1.5 wt % to about 6.0 wt %, or about 1.5 wt % to about 5.5 wt %, or about 1.5 wt % to about 5.0 wt %, or about 1.5 wt % to about 4.5 wt %, or about 1.5 wt % to about 4.0 wt %, or about 1.5 wt % to about 3.5 wt %, or about 1.5 wt % to about 3.0 wt %, or about 1.5 wt % to about 2.5 wt %, or about 1.5 wt % to about 2.0 wt %, or about 1.75 wt % to about 7.0 wt %, or about 1.75 wt % to about 6.5 wt %, or about 1.75 wt % to about 6.0 wt %, or about 1.75 wt % to about 5.5 wt %, or about 1.75 wt % to about 5.0 wt %, or about 1.75 wt % to about 4.5 wt %, or about 1.75 wt % to about 4.0 wt %, or about 1.75 wt % to about 3.5 wt %, or about 1.75 wt % to about 3.0 wt %, or about 1.75 wt % to about 2.5 wt %, or about 1.75 wt % to about 2.0 wt %, or about 2.0 wt % to about 7.0 wt %, or about 2.0 wt % to about 6.5 wt %, or about 2.0 wt % to about 6.0 wt %, or about 2.0 wt % to about 5.5 wt %, or about 2.0 wt % to about 5.0 wt %, or about 2.0 wt % to about 4.5 wt %, or about 2.0 wt % to about 4.0 wt %, or about 2.0 wt % to about 3.5 wt %, or about 2.0 wt % to about 3.0 wt %, or about 2.0 wt % to about 2.5 wt %, or about 2.5 wt % to about 7.0 wt %, or about 2.5 wt % to about 6.5 wt %, or about 2.5 wt % to about 6.0 wt %, or about 2.5 wt % to about 5.5 wt %, or about 2.5 wt % to about 4.5 wt %, or about 2.5 wt % to about 4.0 wt %, or about 2.5 wt % to about 3.5 wt %, or about 2.5 wt % to about 3.0 wt %, or about 3.0 wt % to about 7.0 wt %, or about 3.0 wt % to about 6.5 wt %, or about 3.0 wt % to about 6.0 wt %, or about 3.0 wt % to about 5.5 wt %, or about 3.0 to about 5.0 wt % or about 3.0 wt % to about 4.5 wt %, or about 3.0 wt % to about 4.0 wt %, or about 3.0 wt % to about 3.5 wt %, or about 3.5 wt % to about 7.0 wt %, or about 3.5 wt % to about 6.5 wt %, or about 3.5 wt % to about 6.0 wt %, or about 3.5 wt % to about 5.5 wt % or about 3.5 wt % to about 4.5 wt %, or about 3.5 wt % to about 4.0 wt %, or about 4.0 wt % to about 7.0 wt %, or about 4.0 wt % to about 6.5 wt %, or about 4.0 wt % to about 6.0 wt %, or about 4.0 wt % to about 5.5 wt %, or about 4.0 to about 5.0 wt % or about 4.0 wt % to about 4.5 wt %, or about 4.5 wt % to about 7.0 wt %, or about 4.5 wt % to about 6.5 wt %, or about 4.5 wt % to about 6.0 wt %, or about 4.5 wt % to about 5.5 wt % or about 4.5 wt % to about 5.0 wt %, or about 5.0 wt % to about 7.0 wt %, or about 5.0 wt % to about 6.5 wt %, or about 5.0 wt % to about 6.0 wt %, or about 5.0 wt % to about 5.5 wt %, or about 5.5 wt % to about 7.0 wt %, or about 5.5 wt % to about 6.5 wt %, or about 5.5 wt % to about 6.0 wt %, or about 6.0 wt % to about 7.0 wt %, or about 6.0 wt % to about 6.5 wt % or about 6.5 wt % to about 7.0 wt %.

The compositions of the present invention may contain at least one non-steroidal anti-inflammatory drugs (NSAIDS). NSAIDs are a group of chemically dissimilar agents that have primary effect of inhibition of prostaglandin's synthesis. NSAIDS that may be part of the compositions of the present invention include but are not limited to acetyl salicylic acid, acetaminophen and ibuprofen which are also used as pain relievers.

The amount of each NSAID compound in the compositions of the present invention may be about 0.5 wt %, 0.6 wt % 0.7 wt %, 0.8 wt %, 0.9 wt %, 1.0 wt %, 1.1 wt % 1.2 wt %, 1.3 wt %, 1.4 wt %, 1.5 wt %, 1.6 wt %, 1.7 wt %, 1.8 wt %, 1.9 wt %, 2.0 wt %, 2.1 wt %, 2.2 wt %, 2.3 wt %, 2.4 wt %, 2.5 wt %, 2.6 wt %, 2.7 wt %, 2.8 wt %, 2.9 wt %, 3.0 wt %, 3.1 wt %, 3.2 wt %, 3.3 wt %, 3.4 wt %, 3.5 wt %, 3.6 wt %, 3.7 wt %, 3.8 wt %, 3.9 wt %, 4.0 wt %, 4.1 wt %, 4.2 wt %, 4.3 wt %, 4.4 wt %, 4.5 wt %, 4.6 wt %, 4.7 wt %, 4.8 wt %, 4.9 wt %, 5.0 wt %, 5.1 wt %, 5.2 wt %, 5.3 wt %, 5.4 wt %, 5.5 wt %, 5.6 wt %, 5.7 wt %, 5.8 wt %, 5.9 wt %, 6.0 wt %, 6.1 wt %, 6.2 wt %, 6.3 wt %, 6.4 wt %, 6.5 wt %, 6.6 wt %, 6.7 wt %, 6.8 wt %, 6.9 wt %, 7.0 wt %.

The amount of each NSAID compound in the compositions of the present invention may be between about 0.1 wt % to about 5.0 wt %, or about 0.1 wt % to about 4.5 wt %, or about 0.1 wt % to about 4.0 wt %, or about 0.1 wt % to about 3.5 wt %, or about 0.1 wt % to about 3.0 wt %, or about 0.1 wt % to about 2.5 wt %, or about 0.1 wt % to about 2.0 wt %, or about 0.1 wt % to about 1.5 wt %, or about 0.1 wt % to about 1.0 wt %, or about 0.1 wt % to about 0.5 wt %, or about 0.1 wt % to about 0.3 wt %, or about 0.3 wt % to about 5.0 wt %, or about 0.3 wt % to about 4.5 wt %, or about 0.3 wt % to about 4.0 wt %, or about 0.3 wt % to about 3.5 wt %, or about 0.3 wt % to about 3.0 wt %, or about 0.3 wt % to about 2.5 wt %, or about 0.3 wt % to about 2.0 wt %, or about 0.3 wt % to about 1.5 wt %, or about 0.3 wt % to about 1.0 wt %, or about 0.3 wt % to about 0.5 wt %, or about 0.5 wt % to about 5.0 wt %, or about 0.5 wt % to about 4.5 wt %, or about 0.5 wt % to about 4.0 wt %, or about 0.5 wt % to about 3.5 wt %, or about 0.5 wt % to about 3.0 wt %, or about 0.5 wt % to about 2.5 wt %, or about 0.5 wt % to about 2.0 wt %, or about 0.5 wt % to about 1.5 wt %, or about 0.5 wt % to about 1.0 wt %, or about 0.7 wt % to about 5.0 wt %, or about 0.7 wt % to about 4.5 wt %, or about 0.7 wt % to about 4.0 wt %, or about 0.7 wt % to about 3.5 wt %, or about 0.7 wt % to about 3.0 wt %, or about 0.7 wt % to about 2.5 wt %, or about 0.7 wt % to about 2.0 wt %, or about 0.7 wt % to about 1.5 wt %, or about 0.7 wt % to about 1.0 wt %, or about 1.0 wt % to about 5.0 wt %, or about 1.0 wt % to about 4.5 wt %, or about 1.0 wt % to about 4.0 wt %, or about 1.0 wt % to about 3.5 wt %, or about 1.0 wt % to about 3.0 wt %, or about 1.0 wt % to about 2.5 wt %, or about 1.0 wt % to about 2.0 wt %, or about 1.0 wt % to about 1.5 wt %, or about 1.25 wt % to about 5.0 wt %, or about 1.25 wt % to about 4.5 wt %, or about 1.25 wt % to about 4.0 wt %, or about 1.25 wt % to about 3.5 wt %, or about 1.25 wt % to about 3.0 wt %, or about 1.25 wt % to about 2.5 wt %, or about 1.25 wt % to about 2.0 wt %, or about 1.25 wt % to about 1.5 wt %, or about 1.5 wt % to about 5.0 wt %, or about 1.5 wt % to about 4.5 wt %, or about 1.5 wt % to about 4.0 wt %, or about 1.5 wt % to about 3.5 wt %, or about 1.5 wt % to about 3.0 wt %, or about 1.5 wt % to about 2.5 wt %, or about 1.5 wt % to about 2.0 wt %, or about 1.75 wt % to about 5.0 wt %, or about 1.75 wt % to about 4.5 wt %, or about 1.75 wt % to about 4.0 wt %, or about 1.75 wt % to about 3.5 wt %, or about 1.75 wt % to about 3.0 wt %, or about 1.75 wt % to about 2.5 wt %, or about 1.75 wt % to about 2.0 wt %, or about 2.0 wt % to about 5.0 wt %, or about 2.0 wt % to about 4.5 wt %, or about 2.0 wt % to about 4.0 wt %, or about 2.0 wt % to about 3.5 wt %, or about 2.0 wt % to about 3.0 wt %, or about 2.0 wt % to about 2.5 wt %, or about 2.5 wt % to about 4.5 wt %, or about 2.5 wt % to about 4.0 wt %, or about 2.5 wt % to about 3.5 wt %, or about 2.5 wt % to about 3.0 wt %.

The compositions of the present invention may contain hydrogen peroxide. The amount of the hydrogen peroxide in the compositions of the present invention may be about 5 wt %, 6 wt % 7 wt %, 8 wt %, 9 wt %, 10 wt %, 11 wt % 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 30 wt %, 31 wt %, 32 wt %, 33 wt %, 34 wt %, 35 wt %, 36 wt %, 37 wt %, 38 wt %, 39 wt %, 40 wt %, 41 wt %, 42 wt %, 43 wt %, 44 wt %, 45 wt %, 46 wt %, 47 wt %, 48 wt %, 49 wt %, 50 wt %, 51 wt %, 52 wt %, 53 wt %, 54 wt %, 55 wt %, 56 wt %, 57 wt %, 58 wt %, 59 wt %, or 60 wt

The amount of hydrogen peroxide in the compositions of the present invention may be between about 1 wt % to about 50 wt %, or about 1 wt % to about 45 wt %, or about 1 wt % to about 40 wt %, or about 1 wt % to about 35 wt %, or about 1 wt % to about 30 wt %, or about 1 wt % to about 25 wt %, or about 1 wt % to about 20 wt %, or about 1 wt % to about 15 wt %, or about 1 wt % to about 10 wt %, or about 5 wt % to about 50 wt %, or about 5 wt % to about 45 wt %, or about 5 wt % to about 40 wt %, or about 5 wt % to about 35 wt %, or about 5 wt % to about 30 wt %, or about 5 wt % to about 25 wt %, or about 5 wt % to about 20 wt %, or about 5 wt % to about 15 wt %, or about 5 wt % to about 10 wt %, or about 10 wt % to about 45 wt %, or about 10 wt % to about 40 wt %, or about 10 wt % to about 35 wt %, or about 10 wt % to about 30 wt %, or about 10 wt % to about 25 wt %, or about 10 wt % to about 20 wt %, or about 10 wt % to about 15 wt %, or about 15 wt % to about 45 wt %, or about 15 wt % to about 40 wt %, or about 15 wt % to about 35 wt %, or about 15 wt % to about 30 wt %, or about 15 wt % to about 25 wt %, or about 15 wt % to about 20 wt %, or about 20 wt % to about 45 wt %, or about 20 wt % to about 40 wt %, or about 20 wt % to about 35 wt %, or about 20 wt % to about 30 wt %, or about 20 wt % to about 25 wt %, or about 25 wt % to about 45 wt %, or about 25 wt % to about 40 wt %, or about 25 wt % to about 35 wt %, or about 25 wt % to about 30 wt %, or about 20 wt % to about 30 wt %, or about 15 wt % to about 30 wt %.

The compositions of the present invention may contain at least one tissue penetrating solvent in which the other compounds of the compositions of the present invention are dissolved. Numerous compounds have been evaluated for penetration enhancing activity, including but not limited to sulphoxides (such as dimethylsulphoxide, DMSO), Azones (e.g. laurocapram), pyrrolidones (for example 2-pyrrolidone, 2P), alcohols and alkanols (ethanol, or decanol), glycols (for example propylene glycol, PG, a common excipient in topically applied dosage forms), surfactants (also common in dosage forms) and terpenes.

The treatment of dermatophytoses would be most appropriate when the selection of antimicrobial agent is based on the identity of the causative agent. For e.g. griseofulvin is effective only for dermatophytic infections, with no activity against Candida spp. and nondermophytic molds. Terbinafine shows fungicidal activity against dermatophytes with a cure rate of 80 to 95% but shows only fungistatic activity against Candida albicans. For nondermatophytic molds infections, the role of terbinafine is not well defined and topical amorolfine lacquer may be effective for select patients.

Recently, molecular biology-based techniques, such as PCR followed by restriction fragment length polymorphism (RFLP), Real time PCR and multiplex PCR assay and DNA sequencing have been adapted for detection of dermatophytes from clinical specimen. These molecular methods have a good potential to directly detect dermatophytes in clinical specimens; however these methods are yet to be standardized for routine clinical laboratories. The present invention relates to methods of determining the fungal and bacterial species in a given sample of a person with onychomycosis and formulating a topical treatment for each patient based on the results of the species determination.

### EXAMPLES

### Example 1

### DNA Sequencing of OMB infection

DNA sequencing methods were used to identify pathogens and estimate the percentage population of those pathogens present in specimens of OMB. Seven patients with OMB were tested to determine the pathogens present in the OMB sample. Table 1 indicates the frequency of pathogens present in the samples.

TABLE 1

| | Detected | Not Detected |
|---|---|---|
| Pathogen | | |
| Acinetobacter baumannii | 0 | 7 |
| Escherichia coli | 0 | 7 |
| Enterobacter aerogenes | 0 | 7 |
| Enterobacter cloacae | 0 | 7 |
| Enterococci faecalis | 1 | 6 |
| Enterococci faecium | 0 | 7 |
| Klebsiella pneumoniae | 0 | 7 |
| Proteus mirabilis | 1 | 6 |
| Pseudomonas aeruginosa | 0 | 7 |
| Serratia marcescens | 0 | 7 |
| Stenotrophonmonas maltophilia | 0 | 7 |
| Methicillin resistant staph aureus | 2 | 5 |
| Staphylococcus aureus | 0 | 7 |
| Panton-Vallentine leucocidin (PVL) gene | 0 | 7 |
| Coagulase negative Staphylococcus | 3 | 4 |
| Methicillin resistant | | |
| Coagulase negative Staphylococcus | 3 | 4 |
| Staphylococcus epidermidis | 2 | 5 |
| Antibiotic resistant | | |
| Aminoglycosides resistance | 1 | 6 |
| Cephalosporin resistance | 4 | 3 |
| Erythromycin-Clindamycin resistance | 5 | 2 |
| Methicillin resistant (staphylococcus) | 4 | 3 |
| Tetracycline resistance | 6 | 1 |
| Vancomycin resistance | 0 | 7 |

In another study the following pathogens were found in specimen at the given percentage:

TABLE 2

| Pathogen | Percentage |
|---|---|
| Staphylococcus epidermis | 82% |
| Cornebacterium kroppenstedii | 8% |
| Propionibacterium granulosum | 3% |
| Staphylococcus pasteuri | 2% |

Within this disclosure, any indication that a feature is optional is intended provide adequate support (e.g., under 35 U.S.C. 112 or Art. 83 and 84 of EPC) for claims that include closed or exclusive or negative language with reference to the optional feature. Exclusive language specifically excludes the particular recited feature from including any additional subject matter. For example, if it is indicated that A can be drug X, such language is intended to provide support for a claim that explicitly specifies that A consists of X alone, or that A does not include any other drugs besides X. “Negative” language explicitly excludes the optional feature itself from the scope of the claims. For example, if it is indicated that element A can include X, such language is intended to provide support for a claim that explicitly specifies that A does not include X. Non-limiting examples of exclusive or negative terms include “only,” “solely,” “consisting of,” “consisting essentially of,” “alone,” “without”, “in the absence of (e.g., other items of the same type, structure and/or function)” “excluding,” “not including”, “not”, “cannot,” or any combination and/or variation of such language.

Similarly, referents such as “a,” “an,” “said,” or “the,” are intended to support both single and/or plural occurrences unless the context indicates otherwise. For example “a dog” is intended to include support for one dog, no more than one dog, at least one dog, a plurality of dogs, etc. Non-limiting examples of qualifying terms that indicate singularity include “a single”, “one,” “alone”, “only one,” “not more than one”, etc. Non-limiting examples of qualifying terms that indicate (potential or actual) plurality include “at least one,” “one or more,” “more than one,” “two or more,” “a multiplicity,” “a plurality,” “any combination of,” “any permutation of,” “any one or more of,” etc. Claims or descriptions that include “or” between one or more members of a group are considered satisfied if one, more than one, or all of the group members are present in, employed in, or otherwise relevant to a given product or process unless indicated to the contrary or otherwise evident from the context.

Where ranges are given herein, the endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While this invention has been particularly shown and described with references to example embodiments thereof, it will be understood by those skilled in the art that the various changes in form and details may be made therein without departing from the scope of the invention encompassed by the appended claims.

1. A composition for the treatment of onychomycosis comprising at least one antibiotic, at least one antifungal agent in a tissue penetrating agent.

2. The composition of claim 1 further comprising a non-steroidal anti-inflammatory agent.

3. The composition of claim 2 wherein the antifungal agents are itraconazole and fluconazole and the antibiotics are vancomycin, levofloxacin and mupirocin.

4. The composition of claim 3 wherein the antifungal agents are itraconazole and fluconazole and the antibiotics are vancomycin, levofloxacin and mupirocin and the non-steroidal anti-inflammatory agent is ibuprofen.

5. The composition of claim 6 wherein the itraconazole is at about 0.5 wt % to about 1.5 wt %, the fluconazole is about 0.5 wt % to about 1.5 wt %, the vancomycin is about 4 wt %to about 5 wt %, the levofloxacin is about 1 wt % to about 3 wt %, the mupirocin is about 3 wt % to about 5 wt % and the ibuprofen is about 1 wt % to about 3 wt % and the tissue penetrating agent is DMSO.

6. The composition of claim 5 wherein the itraconazole is at about 1 wt %, the fluconazole is about 1 wt %, the vancomycin is about 5 wt %, the levofloxacin is about 2 wt %, the mupirocin is about 4 wt % and the ibuprofen is about 2 wt % and the tissue penetrating agent is DMSO.

7. A method treating onychomycosis comprising administering to the nail(s) of a patient having onychomycosis the composition of claim 5.

8. The method of claim 7 wherein the administration is daily

9. The method of claim 8 wherein the duration of the treatment is at least a week.

10. A method of determining an optimal formulation for treating onychomycosis in an individual patient comprising:
a) testing a nail sample that is infected;
b) determining the pathogen species present in the infection; and
c) formulating a composition for treating onychomycosis based on the types of fungal and/or bacterial species present in the sample.

11. The method of claim 10 wherein the sample is tested by real time PCR or multiplex PCR assay.

12. The method of claim 10 wherein the sample is tested by DNA sequencing.

13. The method of claim 12 wherein the sample tested by DNA sequencing can determine the relative percentage of pathogens in the sample.

14. A composition for the treatment of onychomycosis comprising hydrogen peroxide and an antibiotic.

15. The composition of claim 14 wherein the antibiotic is either vancomycin or clindamycin.

16. The composition of claim 15 wherein the vancomycin is present in an amount from 1-5 wt %.

17. The composition of claim 15 wherein the clindamycin is present in an amount from 1-5 wt %.

18. The composition of claim 14 wherein the hydrogen peroxide is present in an amount of about 20 to about 30%.

19. The composition of claim 18 wherein the vancomycin is present in an amount from 1-5 wt %.

20. The composition of claim 18 wherein the clindamycin is present in an amount from 1-5 wt %.
